(19) United States
(12) Patent Application Publication
(10) Pub. No.: US 2025/0084031 A1
(43) Pub. Date: Mar. 13, 2025

(54) METHOD OF PREPARING GLUCOSE-DERIVED ADIPONITRILE AND A METHOD OF DEHYDRATING BIOMASS-DERIVED AMIDE COMPOUND

(71) Applicants: HYUNDAI MOTOR COMPANY, Seoul (KR); KIA CORPORATION, Seoul (KR); Seoul National University R&DB Foundation, Seoul (KR)

(72) Inventors: Chaehwan Hong, Seoul (KR); Dongwon Yoo, Seoul (KR); Hyunwoo Choi, Seoul (KR); Gukhee Yim, Seoul (KR)

(73) Assignees: HYUNDAI MOTOR COMPANY, Seoul (KR); KIA CORPORATION, Seoul (KR); Seoul National University R&DB Foundation, Seoul (KR)

(21) Appl. No.: 18/520,847

(22) Filed: Nov. 28, 2023

(30) Foreign Application Priority Data
Sep. 8, 2023 (KR) 10-2023-0119366

(51) Int. Cl. C07C 255/04; C07C 255/27

(52) U.S. Cl. CPC C07C 255/04; C07C 255/27; C07C 2521/08; C07C 2523/22; C07C 2523/26; C07C 2523/28

(57) ABSTRACT

Disclosed is a method of preparing biomass-derived adiponitrile including preparing adiponitrile by subjecting biomass-derived adipamide to dehydration using a catalyst in a medium. The catalyst is configured such that a metal oxide is supported on a carrier containing porous silica with a pore diameter in a range of 5 nm to 15 nm, and the metal is molybdenum, chromium, vanadium, or combinations thereof.

### CROSS-REFERENCE TO RELATED APPLICATION

This application claims, under 35 U.S.C. § 119 (a), the benefit of and priority to Korean Patent Application No. 10-2023-0119366, filed on Sep. 8, 2023, the entire contents of which are incorporated herein by reference.

### BACKGROUND

### (a) Technical Field

The present disclosure relates to a method of preparing glucose-derived adiponitrile and a method of dehydrating a biomass-derived amide compound.

### (b) Background Art

Recently, development of future resource biomass-related processing technology is underway worldwide. Materials related to interior, exterior, and chassis parts used in vehicles include polypropylene, nylon, polycarbonate, acrylonitrile butadiene styrene, and the like. Among these materials, polypropylene and nylon are mainly used, and when highly versatile nylon is able to be manufactured using technology based on biomass, a significant ripple effect may be expected.

Nylon 66 is in high demand in various fields due to unique properties thereof, but the production process thereof from biomass is insufficient. Therefore, developing a process of producing biomass-derived nylon 66 is advantageous from economic and environmental perspectives and may also contribute to carbon reduction.

Nylon 66 may be prepared by dehydration polymerization of hexamethylenediamine and adipic acid. Adipic acid, serving as a monomer, may currently be produced from benzene obtained during crude oil refining through a chemical synthesis process using cyclohexanone as an intermediate. Also, hexamethylenediamine may be converted from adiponitrile prepared by subjecting butadiene obtained during crude oil refining to chemical reaction with hydrogen cyanide. These processes may cause various problems, such as oil price instability, use of hydrogen cyanide corresponding to a toxic material, and the like. Thus, there is a need for a synthesis method using plant resources, particularly biomass, rather than crude oil-based processes.

### SUMMARY OF THE DISCLOSURE

The present disclosure has been made keeping in mind the problems encountered in the related art and is intended to provide a method of preparing glucose-derived adiponitrile and a method of dehydrating a biomass-derived amide compound, which lower dependence on petroleum-based raw materials, minimize environmental pollution, and satisfy carbon reduction.

In addition, the present disclosure is intended to lower dependence on petroleum-based raw materials and reduce carbon generation in producing nylon 66 resin and other industrial resins using biomass-derived adiponitrile, cyano group-containing compounds, etc.

The objects of the present disclosure are not limited to the foregoing. The objects of the present disclosure are able to be understood through the following description and to be realized by the means described in the claims and combinations thereof.

An aspect of the present disclosure provides a method of preparing glucose-derived adiponitrile, including: (a) preparing adipamide by sequentially subjecting a glucose-derived glucaric acid salt to terminal amine group substitution, deoxydehydration, and hydrogenation; and (b) preparing adiponitrile by subjecting the adipamide prepared in step (a) to dehydration using a catalyst in a medium. In particular, the catalyst comprises a metal oxide supported on a carrier containing porous silica with a pore diameter in a range of 5 nm to 15 nm, wherein the metal may include molybdenum, chromium, vanadium, or combinations thereof.

Here, step (a) may include: (a-1) preparing a glucaric acid salt by mixing glucose, oxygen, a basic solution, and an acidic solution and carrying out a reaction; (a-2) preparing glucaramide by mixing the glucaric acid salt prepared in step (a-1) with an ammonia solution and a solvent and carrying out a reaction for a predetermined period of time; (a-3) preparing 2,4-hexadienediamide by subjecting the glucaramide prepared in step (a-2) to the deoxydehydration in the presence of a metal catalyst; and (a-4) preparing the adipamide by subjecting the 2,4-hexadienediamide prepared in step (a-3) to hydrogenation.

Here, step (a-1) may include: (a-1-1) injecting oxygen gas into an aqueous glucose solution and carrying out a reaction at a predetermined temperature; (a-1-2) adding the basic solution containing an alkali metal or alkaline earth metal to the result of step (a-1-1); (a-1-3) adding the acidic solution to the result of step (a-1-2); and (a-1-4) recovering the glucaric acid salt by filtering a precipitate obtained after allowing the result of step (a-1-3) to stand for a predetermined period of time.

Here, step (a-2) may include: (a-2-1) removing cations by adding an acidic solution to a mixture including the glucaric acid salt prepared in step (a-1) and a solvent and carrying out a reaction; and (a-2-2) recovering the precipitated glucaramide by adding an ammonia solution to the result of step (a-1-1) and adding an alcohol-based material thereto.

The metal catalyst in step (a-3) may include one or more of rhenium oxide, ammonium perrhenate, or LₓReO_{y}, in which L is C1-C5 alkoxy, C1-C5 substituted or unsubstituted alkyl, phosphine, phenylsilyl, halogen, or amine, and x and y are each independently an integer in a range of 1 to 3.

Also, step (a-4) may be performed by mixing the 2,4-hexadienediamide prepared in step (a-3) with hydrogen and a hydrogenation catalyst, wherein the hydrogenation catalyst may include platinum or palladium supported on a carrier, silica, alumina, aluminum, or combinations thereof.

The medium in step (b) may include a benzene derivative in which at least one hydrogen atom is substituted with a C1-C3 alkyl group, benzene, C1-C5 primary alcohol, C1-C5 secondary alcohol, or combinations thereof.

The amount of metal based on the total amount of the catalyst in step (b) may be in a range of 1 wt % to 30 wt %.

The mixing weight ratio of adipamide to the catalyst in step (b) may be 1:0.05 to 1:0.8.

Also, step (b) may be performed in an ambient atmosphere at a temperature in a range of 150° C. to 190° C. for 3 hours to 72 hours.

The catalyst in step (b) may be obtained by: (i) heat treating a carrier containing porous silica with a pore diameter in range of 5 nm to 15 nm at a temperature in a range of 600° C. to 800° C.; and (ii) adding a metal precursor to the result of step (i) and performing heat treatment at a temperature in a range of 400° C. to 600° C., wherein the metal may include molybdenum, chromium, vanadium, or combinations thereof.

The adiponitrile may have 50 wt % or more of bio-based carbon according to ASTM D6866.

Another aspect provides a method of preparing biomass-derived adiponitrile, including (b′) preparing adiponitrile by subjecting adipamide having 50 wt % or more of bio-based carbon according to ASTM D6866 based on the total amount of carbon to dehydration using a catalyst in a medium, wherein the catalyst may be configured such that a metal oxide is supported on a carrier containing porous silica with a pore diameter in a range of 5 nm to 15 nm, and wherein the metal may include molybdenum, chromium, vanadium, or combinations thereof.

Still another aspect provides a method of dehydrating a biomass-derived compound, including (b″) preparing a second compound by subjecting a first compound having 50 wt % or more of bio-based carbon according to ASTM D6866 based on the total amount of carbon to dehydration using a catalyst in a medium, wherein the catalyst may be configured such that a metal oxide is supported on a carrier containing porous silica with a pore diameter in a range of 5 nm to 15 nm, wherein the metal include molybdenum, chromium, vanadium, or combinations thereof, wherein the first compound may include at least one amide group, and wherein the second compound may include at least one cyano group.

The first compound may be a compound represented by Chemical Formula 1 below, and the second compound may be a compound represented by Chemical Formula 2 below.

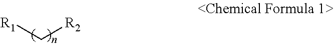

In Chemical Formula 1, R₁ and R₂ each independently contain an amide group, and n is an integer from 1 to 8.

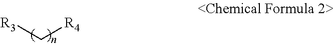

In Chemical Formula 2, R₃ and R₄ each independently contain a cyano group or an amide group, but contain at least one cyano group, and n is an integer from 1 to 8.

The first compound may include adipamide, glutaramide, heptanediamide, or combinations thereof, and the second compound may include adiponitrile, 5-cyanopentanamide, glutaronitrile, 4-cyanobutanamide, heptanedinitrile, 6-cyanohexanamide, or combinations thereof.

### BRIEF DESCRIPTION OF THE DRAWINGS

The above and other features of the present disclosure are now described in detail referring to certain exemplary embodiments thereof illustrated in the accompanying drawings, which are given hereinbelow by way of illustration only, and thus are not limitative of the present disclosure, and wherein:

FIG. 1 is a flowchart schematically showing an example of a process of preparing glucose-derived adiponitrile;

FIG. 2 is a flowchart schematically showing an example of a process of dehydrating a biomass-derived compound;

FIG. 3 schematically shows an example of the process of preparing glucose-derived adiponitrile;

FIG. 4 schematically shows an example of the process of dehydrating biomass-derived adipamide;

FIG. 5 shows transmission electron microscope images of catalysts of Comparative Example 1 and Example 1;

FIG. 6 shows spectral results of nuclear magnetic resonance analysis of adipamide; and

FIG. 7 shows spectral results of nuclear magnetic resonance analysis of the products of Examples.

### DETAILED DESCRIPTION

The above and other objects, features, and advantages of the present disclosure are more clearly understood from the following embodiments taken in conjunction with the accompanying drawings. However, the present disclosure is not limited to the embodiments disclosed herein and may be modified into different forms. These embodiments are provided to thoroughly explain the disclosure and to sufficiently transfer the spirit of the present disclosure to those skilled in the art.

Throughout the drawings, the same reference numerals refer to the same or like elements. For the sake of clarity of the present disclosure, the dimensions of structures are depicted as being larger than the actual sizes thereof. Although terms such as “first,” “second,” etc. may be used herein to describe various elements, these elements are not to be limited by these terms. These terms are only used to distinguish one element from another element. For instance, a “first” element discussed below could be termed a “second” element without departing from the scope of the present disclosure. Similarly, the “second” element could also be termed a “first” element. As used herein, the singular forms are intended to include the plural forms as well, unless the context clearly indicates otherwise.

The terms “comprise,” “include,” “have,” etc., when used in this specification, specify the presence of stated features, integers, steps, operations, elements, components, or combinations thereof, but do not preclude the presence or addition of one or more other features, integers, steps, operations, elements, components, or combinations thereof. Also, when an element such as a layer, film, area, or sheet is referred to as being “on” another element, it may be directly on the other element, or intervening elements may be present therebetween. Similarly, when an element such as a layer, film, area, or sheet is referred to as being “under” another element, it may be directly under the other element, or intervening elements may be present therebetween.

Unless otherwise specified, all numbers, values, and/or representations that express the amounts of components, reaction conditions, polymer compositions, and mixtures used herein are to be taken as approximations including various uncertainties affecting measurement that inherently occur in obtaining these values, among others, and thus should be understood to be modified by the term “about” in all cases. Furthermore, when a numerical range is disclosed in this specification, the range is continuous, and includes all values from the minimum value of said range to the maximum value thereof, unless otherwise indicated. Moreover, when such a range pertains to integer values, all integers including the minimum value to the maximum value are included, unless otherwise indicated.

Currently available processes of preparing crude oil-based compounds may cause problems such as oil price instability, use of toxic materials, generation of environmental pollution byproducts, and the like, so attempts are being made to replace the same with biomass-based processing technology. A method of preparing adiponitrile is disclosed herein using plant resources such as glucose or biomass and a method of dehydrating an amide compound, which are expected to lower crude oil dependence and minimize generation of environmental pollution byproducts.

### Method of Preparing Glucose-Derived Adiponitrile

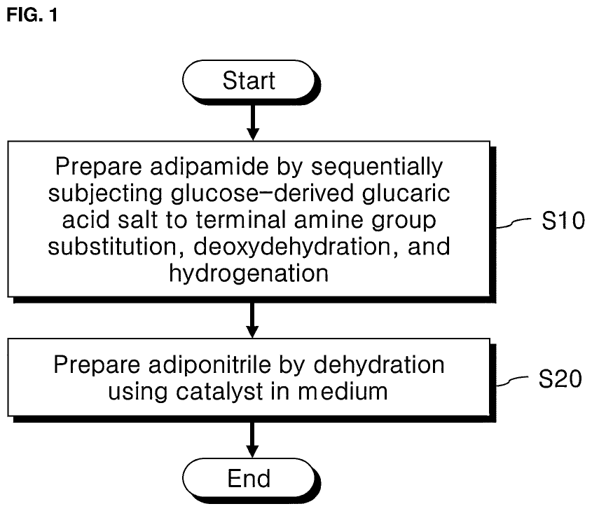

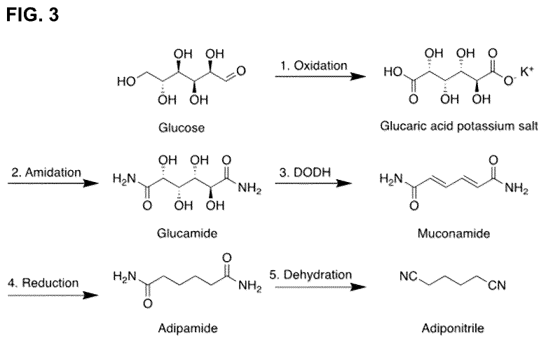

FIG. 1 is a flowchart schematically showing the process of preparing glucose-derived adiponitrile, and FIG. 3 is a flowchart schematically showing the process of preparing adiponitrile and hexamethylenediamine from glucose. Referring thereto, the method of preparing glucose-derived adiponitrile may include: preparing adipamide by sequentially subjecting a glucose-derived glucaric acid salt to terminal amine group substitution, deoxydehydration, and hydrogenation (S10); and preparing adiponitrile by subjecting the adipamide prepared in step (a) to dehydration using a catalyst in a medium (S20).

The catalyst may be configured such that a metal oxide is supported on a carrier containing porous silica with a pore diameter in a range of 5 nm to 15 nm.

The metal may include molybdenum, chromium, vanadium, or combinations thereof.

The glucose may be obtained from plant sources or may be mixed with normal glucose.

Specifically, step (a) (S10) may include: (a-1) preparing a glucaric acid salt by mixing glucose, oxygen, a basic solution, and an acidic solution and carrying out a reaction; (a-2) preparing glucaramide by mixing the glucaric acid salt prepared in step (a-1) with an ammonia solution and a solvent and carrying out a reaction for a predetermined period of time; (a-3) preparing 2,4-hexadienediamide by subjecting the glucaramide prepared in step (a-2) to deoxydehydration in the presence of a metal catalyst; and (a-4) preparing adipamide by subjecting the 2,4-hexadienediamide prepared in step (a-3) to hydrogenation.

Specifically, step (a-1) may include: (a-1-1) injecting oxygen gas into an aqueous glucose solution and carrying out a reaction at a predetermined temperature; (a-1-2) adding a basic solution containing an alkali metal or alkaline earth metal to the result of step (a-1-1); (a-1-3) adding an acidic solution to the result of step (a-1-2); and (a-1-4) recovering the glucaric acid salt by filtering a precipitate obtained after allowing the result of step (a-1-3) to stand for a predetermined period of time.

The oxygen gas in step (a-1-1) may be added into the aqueous glucose solution using an injector, thereby generating oxygen gas bubbles in the aqueous solution.

In step (a-1-1), oxidation may be performed by adding sodium nitrite to a mixture of glucose and nitric acid.

Step (a-1-1) may be performed at a temperature in a range of 40° C. to 60° C. If the temperature is lower than 40° C., reactivity may be decreased, whereas if the temperature is higher than 60° C., unnecessary byproducts may be generated.

The basic solution containing an alkali metal or alkaline earth metal in step (a-1-2) may be exemplified by a basic solution including potassium hydroxide, potassium carbonate, calcium hydroxide, calcium carbonate, etc. Here, the basic solution containing potassium cations may form potassium glucarate, and the basic solution containing calcium cations may form calcium glucarate.

In step (a-1-2), a catalyst may be further added. The catalyst may be configured such that palladium, platinum, or the like is supported on activated carbon.

The pH of the result of step (a-1-2) may be in a range of 9 to 10.

Step (a-1-2) may be performed at room temperature in a range of 15° C. to 30° C.

The acidic solution in step (a-1-3) may include sulfuric acid, hydrochloric acid, phosphoric acid, nitric acid, etc., and for example, nitric acid may be used.

The pH of the result of step (a-1-3) may be in a range of 3 to 4.

In step (a-1-4), the result may be allowed to stand for 6 to 24 hours, and the resulting precipitate may be washed with an alcohol-based material.

Through step (a-1), a glucaric acid salt may be obtained from glucose.

Step (a-2) may include: (a-2-1) removing cations by adding an acidic solution to a mixture including the glucaric acid salt prepared in step (a-1) and a solvent and carrying out a reaction; and (a-2-2) recovering the precipitated glucaramide by adding an ammonia solution to the result of step (a-1-1) and adding an alcohol-based material thereto.

The solvent in step (a-2-1) may be primary alcohol or secondary alcohol. For example, methanol may be used.

The acidic solution in step (a-2-1) may serve to remove metal cations from the glucaric acid salt, and may include sulfuric acid, hydrochloric acid, phosphoric acid, para-toluene sulfonic acid, Amberlyst 15, Amberlite 200, etc. For example, sulfuric acid may be used.

Step (a-2-2) may include thoroughly removing the solvent used in the previous step by evaporation. After removal of the solvent, an ammonia solution may be added at a temperature in a range of −5° C. to 5° C. and mixed for 2 to 4 hours, an alcohol-based material may be added and mixed, and the resulting mixture may be allowed to stand for 10 to 60 minutes.

The concentration of the ammonia solution in step (a-2-2) may be in a range of 25 wt % to 30 wt %.

The alcohol-based material in step (a-2-2) may be exemplified by ethanol.

Through step (a-2), the terminal of the glucaric acid salt may be substituted with an amine group (—NH₂), and glucaramide may be obtained.

In step (a-3), a metal catalyst may be added to a mixture including the glucaramide and a solvent. The solvent may be C1-C5 primary alcohol or secondary alcohol. For example, the solvent may be butanol, 3-pentanol, or 1-heptanol.

The metal catalyst in step (a-3) may include one or more of rhenium oxide, ammonium perrhenate, or LₓReO_{y}, wherein L may be a C1-C5 alkoxy, C1-C5 substituted or unsubstituted alkyl, phosphine, phenylsilyl, halogen, or amine, and each of x and y may be independently an integer of 1 to 3. For example, the metal catalyst may be ammonium perrhenate.

Step (a-3) may be performed at a temperature in a range of 120° C. to 150° C., and may be carried out under reflux stirring for 12 to 24 hours. Under these conditions, 2,4-hexadienediamide may be obtained in good yield.

Step (a-3) may be performed using a Dean-Stark apparatus.

Through step (a-3), deoxydehydration (DODH) may be carried out, and the hydroxyl group of glucaramide may be removed to obtain 2,4-hexadienediamide. Referring to FIG. 3, 2,4-hexadienediamide may also be called muconamide.

Step (a-4) may be performed by mixing the 2,4-hexadienediamide prepared in step (a-3) with hydrogen and a hydrogenation catalyst. The hydrogenation catalyst may include platinum or palladium supported on a carrier, silica, alumina, aluminum, or combinations thereof.

Hydrogen in step (a-4) may be introduced at a pressure in a range of 1 bar to 30 bar.

The reaction in step (a-4) may be carried out at a temperature in a range of 50° C. to 70° C. If the temperature is lower than 50° C., hydrogenation reactivity may decrease, whereas if the temperature exceeds 70° C., preparation efficiency may decrease due to byproducts.

The catalyst in step (a-4) may be, for example, a platinum catalyst supported on an activated carbon carrier, or a palladium catalyst supported on an activated carbon carrier. The amount of noble metal in the catalyst may be in a range of 5 wt % to 12 wt %.

By the hydrogenation in step (a-4), the double bond of 2,4-hexadienediamide may be converted into a single bond, and adipamide may be obtained.

The medium in step (b) (S20) may include a benzene derivative in which at least one hydrogen atom is substituted with a C1-C3 alkyl group, benzene, C1-C5 primary alcohol, C1-C5 secondary alcohol, or combinations thereof. Examples of the medium may include mesitylene, toluene, 3-pentanol, butanol, and 1-heptanol. In one particular example, the medium includes mesitylene.

The catalyst in step (b) (S20) may be configured such that molybdenum oxide or chromium oxide, (e.g., molybdenum oxide (MoOₓ, x=2 or 3)), is supported on a carrier containing porous silica with a pore diameter (or maximum pore length) in a range of 5 nm to 15 nm or in a range of 6 nm to 12 nm. If the pore diameter of the catalyst is less than 5 nm, dehydration may not be effectively promoted, whereas if the pore diameter of the catalyst exceeds 15 nm, preparation yield may decrease.

The catalyst in step (b) (S20) may have pores with a volume in a range of 0.8 cm³/g to 2.2 cm³/g, or in a range of 1.0 cm³/g to 1.8 cm³/g.

The carrier of the catalyst in step (b) (S20) may be, for example, SBA-15 (Santa Barbara Amorphous-15) treated as in steps (i) and (ii) below.

The catalyst in step (b) (S20) may be obtained by: heat treating a carrier

containing porous silica with a pore diameter in a range of 5 nm to 15 nm at a temperature in a range of 600° C. to 800° C.; and adding a metal precursor to the result of step (i) and performing heat treatment at a temperature in a range of 400° C. to 600° C.

The heat treatment in step (i) may be performed for 0.5 to 3 hours.

The heat treatment in step (ii) may be performed for 1 hour to 10 hours.

The metal precursor in step (ii) may be a molybdenum precursor, a chromium precursor, or a vanadium precursor. Examples thereof may include ammonium heptamolybdate ((NH₄)₆·Mo₇O₂₄·4H₂O), molybdenum oxide, molybdenum acetate, molybdenum acetylacetonate, molybdenum oxytriethoxide, chromium nitrate, chromium acetate hydroxide, chromium oxide, ammonium metavanadate, vanadium oxide, vanadium acetylacetonate, and the like. In one particular example, ammonium heptamolybdate tetrahydrate is used.

Through steps (i) and (ii), the catalyst effective for dehydration of glucose-derived adipamide and a biomass-derived amide compound may be prepared.

The amount of metal based on the total amount of the catalyst in step (b) (S20) may be in a range of 1 wt % to 30 wt %, or in a range of 3 wt % to 20 wt %. If the amount of metal of the catalyst is less than 1 wt %, catalytic activity may deteriorate, whereas if it is greater than 30 wt %, the efficiency of the catalyst may deteriorate compared to the amount of metal used.

The mixing weight ratio of adipamide to the catalyst in step (b) (S20) may be in a range of 1:0.05 to 1:0.8 or in a range of 1:0.2 to 1:0.7.

Step (b) (S20) may be performed in an ambient atmosphere at a temperature in a range of 150° C. to 190° C. for 3 hours to 72 hours, at a temperature in a range of 160° C. to 180° C. for 12 hours to 48 hours, or at a temperature in a range of 168° C. to 177° C. for 15 to 40 hours. If the temperature in step (b) is lower than 150° C., preparation yield may decrease, whereas if the temperature is higher than 190° C., side reactions may occur due to excessive temperature conditions.

Step (b) (S20) may be performed in a Dean-Stark apparatus or the like.

The adiponitrile obtained in step (b) (S20) may have 50 wt % or more, 80 wt % or more, or 99 wt % or more of bio-based carbon according to ASTM D6866. The amount of bio-based carbon may be 100 wt % or less. The bio-based carbon may be included from biomass-derived compounds, glucose, etc., and may be partially or substantially entirely included in the amount described above.

The method of preparing glucose-derived adiponitrile is capable of easily converting glucose-derived adipamide into adiponitrile and achieving good yield.

### Method of Preparing Biomass-Derived Adiponitrile

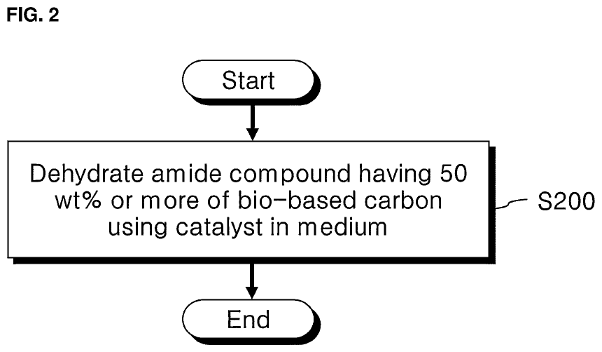

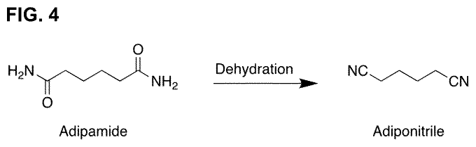

FIG. 2 is a flowchart schematically showing the process of dehydrating a biomass-derived compound, and FIG. 4 schematically shows an example of the process of dehydrating biomass-derived adipamide. Referring thereto, the method of preparing biomass-derived adiponitrile may include: (b′) preparing adiponitrile by subjecting adipamide having 50 wt % or more of bio-based carbon according to ASTM D6866 based on the total amount of carbon to dehydration using a catalyst in a medium.

The catalyst may be configured such that a metal oxide is supported on a carrier containing porous silica with a pore diameter in a range of 5 nm to 15 nm.

The metal may include molybdenum, chromium, vanadium, or combinations thereof.

The amount of bio-based carbon in the adipamide and adiponitrile in step (b′) may be 50 wt % or more, 80 wt % or more, or 99 wt % or more. The amount of bio-based carbon may be 100 wt % or less. The amount of bio-based carbon may be substantially the same as the amount of bio-carbon in the adiponitrile in step (b) described above.

The adipamide in step (b′) may be prepared through step (a) described above, or may be prepared by other known methods, and may include a predetermined amount or more of bio-based carbon.

The medium, catalyst, dehydration conditions, expected effects, etc. in step (b′) may be substantially the same as those in step (b) described above.

### Method of Dehydrating Biomass-Derived Compound

FIG. 2 is a flowchart schematically showing the process of dehydrating a biomass-derived compound, and FIG. 4 schematically shows an example of the process of dehydrating biomass-derived adipamide. Referring thereto, the method of dehydrating a biomass-derived compound may include: (b″) preparing a second compound by subjecting a first compound having 50 wt % or more of bio-based carbon according to ASTM D6866 based on the total amount of carbon to dehydration using a catalyst in a medium.

The catalyst may be configured such that a metal oxide is supported on a carrier containing porous silica with a pore diameter in a range of 5 nm to 15 nm.

The metal may include molybdenum, chromium, vanadium, or combinations thereof.

The first compound may contain at least one amide group (—CONH₂).

The second compound may contain at least one cyano group (—CN).

The amount of bio-based carbon in the first compound and the second compound may be 50 wt % or more, 80 wt % or more, or 99 wt % or more. The amount of bio-based carbon may be 100 wt % or less. The amount of bio-based carbon may be substantially the same as the amount of bio-carbon in the adiponitrile in step (b) described above.

The first compound may be a compound containing at least one amide group at a C2-C8 straight or branched alkylene terminal.

The second compound may be a compound containing at least one cyano group at a C2-C8 straight or branched alkylene terminal.

The first compound in step (b″) may be a compound represented by Chemical Formula 1 below.

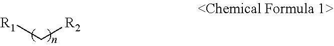

In Chemical Formula 1, R₁ and R₂ each independently contain an amide group, and n is an integer from 1 to 8.

The second compound in step (b″) may be a compound represented by Chemical Formula 2 below.

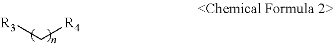

In Chemical Formula 2, R₃ and R₄ each independently contain a cyano group or an amide group, but contain at least one cyano group, and n is an integer from 1 to 8.

The first compound may include adipamide, glutaramide, heptanediamide (pimelamide), or combinations thereof.

The second compound may include adiponitrile, 5-cyanopentanamide, glutaronitrile, 4-cyanobutanamide, heptanedinitrile, 6-cyanohexanamide, or combinations thereof.

The medium, catalyst, dehydration conditions, expected effects, etc. in step (b″) may be substantially the same as those in step (b) described above.

### Catalyst for Dehydration of Biomass-Derived Amide Compound

A catalyst for dehydration of a biomass-derived amide compound may include a carrier containing porous silica with a pore diameter in a range of 5 nm to 15 nm and a metal oxide supported on the carrier.

The metal may include molybdenum, chromium, vanadium, or combinations thereof.

The catalyst carrier, supported metal oxide, use, preparation method, and dehydration treatment method using the catalyst may be substantially the same as those described above.

A better understanding of the present disclosure may be obtained through the following examples and comparative examples. However, these examples are not construed as limiting the technical spirit of the present disclosure.

### Supporting of Catalyst

Catalysts were prepared under different conditions for the pore diameter of the carrier, the amount of supported metal, and the supported metal material according to Examples and Comparative Examples.

A carrier was calcined in an ambient atmosphere at a temperature of 700° C. for 1 hour.

The calcined carrier was impregnated with a metal precursor and adsorbed, followed by heat treatment at a temperature of 500° C. for 3 hours, thereby preparing a catalyst.

When the metal to be supported was molybdenum, heptamolybdate tetrahydrate was used.

### Preparation of Glucose-Derived Adipamide

Adipamide was prepared as follows.
(a-1-1) Oxygen gas was added to an aqueous glucose solution using a needle injector, and oxidation was carried out at a temperature maintained at 50° C.
(a-1-2) The temperature was decreased to 25° C., and 45 wt % potassium hydroxide and a palladium-supported activated carbon catalyst were added to adjust pH to 9-10 to form potassium glucarate.
(a-1-3) 70 wt % nitric acid was added to adjust pH to 3-4 to prepare an acidic reaction product.
(a-1-4) The reaction product was allowed to stand at room temperature for 12 hours, and the resulting solid precipitate was filtered and then washed with methanol to prepare potassium glucarate with high purity.
(a-2-1) Sulfuric acid was added to a mixture of the prepared potassium glucarate and methanol, maintained for 18 hours, and then cooled to a temperature of 25° C. to remove precipitated potassium sulfate.
(a-2-2) Methanol was thoroughly removed by evaporation, the result was maintained in a bath at 0° C., and an aqueous ammonia solution was added thereto, followed by mixing for 3 hours. Then, ethanol was further added thereto, and the result was mixed for 10 minutes and allowed to stand for 10 minutes to prepare solid glucaramide.
(a-3) A mixture of the prepared glucaramide with a butanol solvent and an ammonium perrhenate catalyst was prepared and then subjected to deoxydehydration under reflux stirring for 18 hours at a temperature of 135° C. in a Dean-Stark apparatus to obtain 2,4-hexadienediamide (muconamide).
(a-4) Hydrogen gas was introduced at a pressure of 15 bar into the 2,4-hexadienediamide mixture, a palladium-supported activated carbon catalyst was added thereto, and hydrogenation was carried out at a temperature of 60° C. to yield adipamide.

### Example 1

### Preparation of Adiponitrile using MoOₓ/SBA-15 Catalyst 1

(b) 0.216 g of the adipamide prepared above was mixed with 20 cc of a mesitylene medium and then placed in a chemical reactor (Dean-Stark). Next, 60 mg of a catalyst with 4 wt % molybdenum based on the total amount thereof in which molybdenum oxide (MoOₓ) was supported on a mesoporous silica SBA-15 (Santa Barbara Amorphous-15) carrier with a pore diameter of 10 nm was prepared and added. Next, adiponitrile was prepared by dehydration treatment in an ambient atmosphere at a temperature of 170° C. for 24 hours.

### Example 2

### Preparation of Adiponitrile using MoOₓ/SBA-15 Catalyst 2

Adiponitrile was prepared under the same conditions as in Example 1, with the exception that the amount of molybdenum in the catalyst was changed to 9 wt %.

### Example 3

### Preparation of Adiponitrile Using MoOₓ/SBA-15 Catalyst 3

Adiponitrile was prepared under the same conditions as in Example 1, with the exception that the amount of molybdenum in the catalyst was changed to 18 wt %.

### Comparative Example 1

### Adipamide Treatment Using MoOₓ/Nonporous Silica Catalyst 1

Adipamide was treated under the same conditions as in Example 1, with the exception that Alfa Aesar product code 044740 nonporous silica was used as the catalyst carrier.

### Comparative Example 2

### Adipamide Treatment Using MoOₓ/Silica-Alumina Catalyst 2

Adipamide was treated under the same conditions as in Example 1, with the exception that silica-alumina was used as the catalyst carrier.

The silica-alumina carrier was prepared as follows.

0.45 mmol (997 μl) of tetraethyl orthosilicate (TEOS) was added to 50 ml of a mixture of ethanol and water at a volume ratio of 1:1. Then, 0.9 mmol (217.3 mg) of

AlCl₃·6H₂O was added so that the molar ratio of silica to alumina was 5:1, and about 0.1 ml of NH₄OH was added so that the pH was 4.0 or less. This mixture was treated at a temperature of 100° C. for 20 hours in a hydrothermal synthesizer, and the resulting solid product was centrifuged and then washed with an ethanol+water solution. After drying at 120° C., heat treatment in an ambient atmosphere at 550° C. for 4 hours in a furnace was performed. The amount of alumina in the carrier thus obtained was 17 wt %.

### Comparative Example 3

### Adipamide Treatment Using MoOₓ/Silica-Alumina Catalyst 3

Adipamide was treated under the same conditions as in Comparative Example 2, with the exception that the molar ratio of silica to alumina in the carrier was changed to 3:1.

### Comparative Example 4

### Adipamide Treatment Using MoOₓ/SBA-15 Catalyst 4

Adipamide was treated under the same conditions as in Example 1, with the exception that the pore diameter of the catalyst carrier was changed to 4 nm and the amount of supported metal was changed to 9 wt %.

The catalyst conditions of Examples and Comparative Examples are summarized in Table 1 below.

TABLE 1

| | | | | Amount of |
|---|---|---|---|---|
| | Pore | | | supported |
| | diameter | | Supported | metal |
| Classification | (nm) | Carrier | material | (wt %) |
| Example 1 | 10 | SBA-15 | MoOₓ | 4 |
| Example 2 | 10 | SBA-15 | MoOₓ | 9 |
| Example 3 | 10 | SBA-15 | MoOₓ | 18 |
| Comparative | — | Silica | MoOₓ | 4 |
| Example 1 | | | | |
| Comparative | — | Silica-alumina | MoOₓ | 4 |
| Example 2 | | | | |
| Comparative | — | Silica-alumina | MoOₓ | 4 |
| Example 3 | | | | |
| Comparative | 4 | SBA-15 | MoOₓ | 9 |
| Example 4 | | | | |

*In MoOₓ, x is 2 or 3

### Test Example 1

### Transmission Electron Microscopy of Catalyst

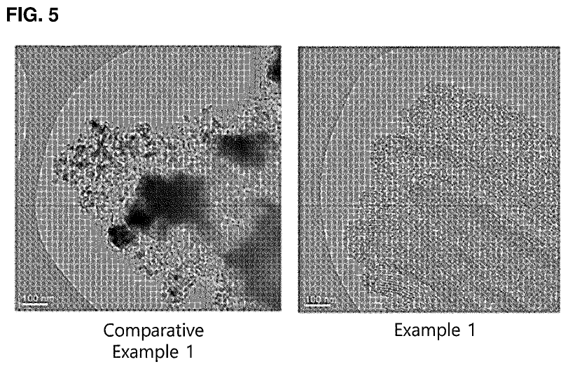

The catalyst of Comparative Example 1 and the catalyst of Example 1 were observed with a transmission electron microscope and the results thereof are shown in FIG. 5. Referring thereto, it was confirmed that molybdenum oxide was uniformly distributed in the mesoporous silica carrier with a pore diameter of 10 nm in Example 1 compared to the silica carrier of Comparative Example 1.

### Test Example 2

### Nuclear Magnetic Resonance (NMR) Analysis and Yield Measurement

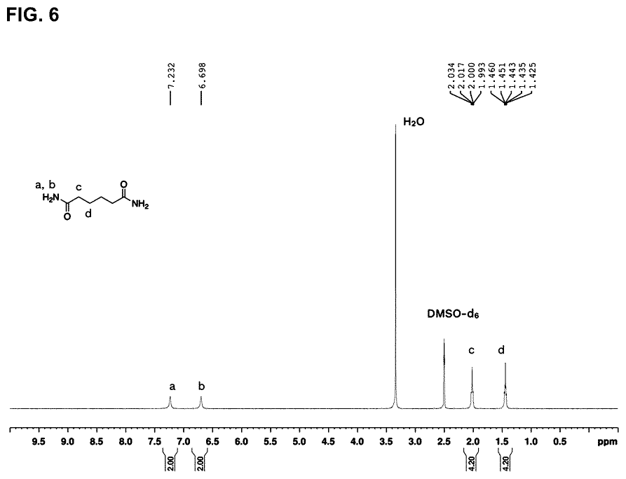

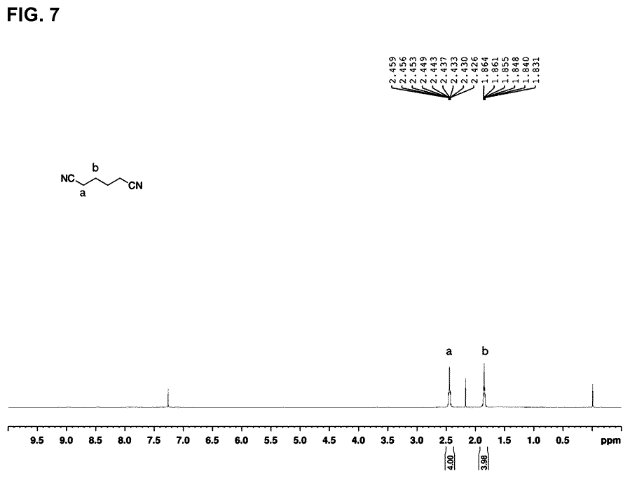

Nuclear magnetic resonance analysis of the materials treated in Comparative Examples and Examples was performed using a Bruker AVIII400 device, and the results thereof are shown in FIGS. 6 and 7 and below.

Adipamide: ¹H NMR (400 MHz, DMSO-d₆) δ 7.27 (s, 2H), 6.72 (s, 2H), 2.02 (m, 4H), 1.44 (m, 4H)

Examples: ¹H NMR (400 MHz, CDCl₃) δ 2.45 (m, 4H), 1.85 (m, 4H)

Referring thereto, based on NMR results of Examples 1 to 3 as shown in FIG. 7, it was confirmed that the adiponitrile material was synthesized.

The adiponitrile preparation yields of the treated materials in Examples and Comparative Examples are shown in Table 2 below.

TABLE 2

| Classification | Yield (%) |
|---|---|
| Example 1 | 16 |
| Example 2 | 34 |
| Example 3 | 54 |
| Comparative Example 1 | 3 |
| Comparative Example 2 | 5 |
| Comparative Example 3 | 5 |
| Comparative Example 4 | 1 |

Referring thereto, Examples using catalysts in which molybdenum was supported on the mesoporous silica carrier with a pore diameter in a range of 5 nm to 15 nm showed good yield, and Comparative Examples in which the pore diameter fell outside the appropriate pore diameter range or other metals and carriers were applied substantially did not prepare adiponitrile or showed low yield.

### Test Example 3

### Measurement of Amount of Bio-Carbon

The amount of bio-carbon in the adiponitrile prepared in Example 1 was measured using accelerator mass spectrometry (AMS) according to ASTM D6866-22 Method B. Accordingly, the amount of bio-carbon in the adiponitrile prepared in Example 1 was confirmed to be 99% or more, substantially 100%, based on the total amount of carbon.

As is apparent from the above description, adiponitrile is prepared using plant resources such as glucose or biomass, and the preparation process thereof is advantageous in view of being simplified and environmentally friendly and reducing carbon emissions compared to crude oil-based processes.

Nylon 66 resin obtained using biomass-derived adiponitrile prepared according to the present disclosure can contribute to carbon reduction when used in manufacture of vehicle part materials.

In addition, resins obtained using a biomass-derived cyano group-containing compound dehydrated according to the present disclosure can contribute to carbon reduction when used in manufacture of various part materials.

The effects of the present disclosure are not limited to the above-mentioned effects. It should be understood that the effects of the present disclosure include all effects that can be inferred from the description of the present disclosure.

Although specific embodiments of the present disclosure have been described, those skilled in the art will appreciate that the present disclosure may be embodied in other specific forms without changing the technical spirit or essential features thereof. Thus, the embodiments described above should be understood to be non-limiting and illustrative in every way.

What is claimed is:

1. A method of preparing glucose-derived adiponitrile, the method comprising:
(a) preparing adipamide by sequentially subjecting a glucose-derived glucaric acid salt to terminal amine group substitution, deoxydehydration, and hydrogenation; and
(b) preparing adiponitrile by subjecting the adipamide to dehydration using a catalyst in a medium,
wherein the catalyst comprises a metal oxide supported on a carrier containing porous silica with a pore diameter in a range of 5 nm to 15 nm, and
wherein a metal of the metal oxide comprises molybdenum, chromium, vanadium, or combinations thereof.

2. The method of claim 1, wherein the preparing of the adipamide comprises:
(a-1) preparing a glucaric acid salt by mixing glucose, oxygen, a basic solution, and an acidic solution and carrying out a reaction;
(a-2) preparing glucaramide by mixing the glucaric acid salt with an ammonia solution and a solvent and carrying out a reaction for a predetermined period of time;
(a-3) preparing 2,4-hexadienediamide by subjecting the glucaramide to the deoxydehydration in presence of a metal catalyst; and
(a-4) preparing the adipamide by subjecting the 2,4-hexadienediamide to hydrogenation.

3. The method of claim 2, wherein the preparing of the glucaric acid salt comprises:
(a-1-1) injecting oxygen gas into an aqueous glucose solution and carrying out a reaction at a predetermined temperature;
(a-1-2) adding the basic solution containing an alkali metal or alkaline earth metal to a result of step (a-1-1);
(a-1-3) adding the acidic solution to a result of step (a-1-2); and
(a-1-4) recovering the glucaric acid salt by filtering a precipitate obtained after allowing a result of step (a-1-3) to stand for a predetermined period of time.

4. The method of claim 2, wherein the preparing of the glucaramide comprises:
(a-2-1) removing cations by adding an acidic solution to a mixture comprising the glucaric acid salt and the solvent and carrying out a reaction; and
(a-2-2) recovering the precipitated glucaramide by adding the ammonia solution to a result of step (a-1-1) and adding an alcohol-based material thereto.

5. The method of claim 2, wherein the metal catalyst comprises one or more of rhenium oxide, ammonium perrhenate, or LₓReO_{y}, in which L is a C1-C5 alkoxy, C1-C5 substituted or unsubstituted alkyl, phosphine, phenylsilyl, halogen, or amine, and x and y are each independently an integer in a range of 1 to 3.

6. The method of claim 2, wherein the preparing of the adipamide is performed by mixing the 2,4-hexadienediamide with hydrogen and a hydrogenation catalyst, and
wherein the hydrogenation catalyst comprises platinum or palladium supported on a carrier, silica, alumina, aluminum, or combinations thereof.

7. The method of claim 1, wherein the medium comprises a benzene derivative in which at least one hydrogen atom is substituted with a C1-C3 alkyl group, benzene, C1-C5 primary alcohol, C1-C5 secondary alcohol, or combinations thereof.

8. The method of claim 1, wherein an amount of the metal based on a total amount of the catalyst is in a range of 1 wt % to 30 wt %.

9. The method of claim 1, wherein a mixing weight ratio of the adipamide to the catalyst is in a range of 1:0.05 to 1:0.8.

10. The method of claim 1, wherein the preparing of the adiponitrile is performed in an ambient atmosphere at a temperature in a range of 150° C. to 190° C. for 3 hours to 72 hours.

11. The method of claim 1, wherein the catalyst is obtained by:
(i) heat treating the carrier containing the porous silica at a temperature in a range of 600° C. to 800° C.; and
(ii) adding a metal precursor to a result of step (i) and performing heat treatment at a temperature in a range of 400° C. to 600° C.

12. The method of claim 1, wherein the adiponitrile has 50 wt % or more of bio-based carbon according to ASTM D6866.

13. A method of preparing biomass-derived adiponitrile, the method comprising:
preparing adiponitrile by subjecting adipamide having 50 wt % or more of bio-based carbon according to ASTM D6866 based on a total amount of carbon to dehydration using a catalyst in a medium, and
wherein the catalyst comprises a metal oxide is supported on a carrier containing porous silica with a pore diameter in a range of 5 nm to 15 nm, and
wherein a metal of the metal oxide comprises molybdenum, chromium, vanadium, or combinations thereof.

14. A method of dehydrating a biomass-derived compound, the method comprising:
preparing a second compound by subjecting a first compound having 50 wt % or more of bio-based carbon according to ASTM D6866 based on a total amount of carbon to dehydration using a catalyst in a medium,
wherein the catalyst is configured such that a metal oxide is supported on a carrier containing porous silica with a pore diameter in a range of 5 nm to 15 nm,
wherein a metal of the metal oxide comprises molybdenum, chromium, vanadium, or combinations thereof,
wherein the first compound contains at least one amide group, and
wherein the second compound contains at least one cyano group.

15. The method of claim 14, wherein the first compound is a compound represented by Chemical Formula 1 below, and the second compound is a compound represented by Chemical Formula 2 below: 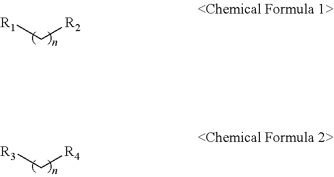
wherein, in Chemical Formula 1, R₁ and R₂ each independently contain an amide group, and n is an integer in a range of 1 to 8, and
wherein, in Chemical Formula 2, R₃ and R₄ each independently contain a cyano group and optionally additionally an amide group, and n is an integer in a range of 1 to 8.

16. The method of claim 15, wherein the first compound comprises adipamide, glutaramide, heptanediamide, or combinations thereof, and
wherein the second compound comprises adiponitrile, 5-cyanopentanamide, glutaronitrile, 4-cyanobutanamide, heptanedinitrile, 6-cyanohexanamide, or combinations thereof.
